(19) United States
(12) Patent Application Publication
(10) Pub. No.: US 2004/0059385 A1
(43) Pub. Date: Mar. 25, 2004

(54) Low frequency assistor for electric-stimulating breast acupuncture point portions of a human body

(76) Inventor: Tsung I. Yu, Taipei (TW)

Correspondence Address: BACON & THOMAS, PLLC, 625 SLATERS LANE, FOURTH FLOOR, ALEXANDRIA, VA, 22314

(21) Appl. No.: 10/352,135

(22) Filed: Jan. 28, 2003

Related U.S. Application Data
(63) Continuation-in-part of application No. 10/253,817, filed on Sep. 25, 2002.

(51) Int. Cl. A61N 1/00

(52) U.S. Cl. 607/2

(57) ABSTRACT

A low frequency assistor serves for electric-stimulating breast acupuncture point portions of a human body. The device comprising at least one pair of positive electrode and negative electrode are connected serially or in parallel on a conductive film or a copper plate or a conductive rubber material so as to be formed as an electrically stimulating piece. In application, the positive electrode and negative electrode are placed on breast acupuncture points of human body. A wire or wireless controller is used to control the at least pair positive electrode and negative electrode of an electrically stimulating piece. The positive electrode and the negative electrode are made as adhering pieces.

The present invention is a continuation in part (CIP) of U.S. Pat. No. 10/253,817, filed at Sep. 25, 2002, which is incorporated into the present invention as a part of the present invention.

### BACKGROUND OF THE INVENTION

(a) Field of the Invention

The present invention relates to health protecting devices, and particularly to a low frequency assistor for electric-stimulating breast acupuncture point portions of a human body, wherein at least one pair of positive electrode and negative electrode are connected serially or in parallel on a conductive film or a copper plate or a conductive rubber material so as to be formed as an electrically stimulating piece; in application, the positive electrode and negative electrode are placed on breast acupuncture points of human body.

(b) Description of the Prior Art

The prior art lower frequency health assistor (for example, massagers, or treater) includes mainly a controller, a silicide electrode pieces or silicide soft glue (called as conductive glue), and bowl shape silicon electrode, etc. The manufacturing cost is high and the timing or place for use is greatly confined. Moreover, the conductivity of the prior art lower frequency health assistor is not preferred. Especially, the user must be coated with silicon glue at the portion to contact the lower frequency health assistor. The glue causes that the user feel uneasy. Besides, the silicide soft glue, and bowl shape silicon electrode are heavy so that the user will be uncomfortable and the lower frequency health assistor can not be hidden well.

### SUMMARY OF THE INVENTION

Accordingly, the primary object of the present invention is to provide a low frequency assistor serves for electric-stimulating breast acupuncture point portions of a human body. The device comprising at least one pair of positive electrode and negative electrode are connected serially or in parallel on a conductive film or a copper plate or a conductive rubber material so as to be formed as an electrically stimulating piece. In application, the positive electrode and negative electrode are placed on breast acupuncture points of human body.

Another object of the present invention is to provide a low frequency assistor for electric-stimulating breast acupuncture point portions of a human body, wherein a wire or wireless controller is used to control the at least pair positive electrode and negative electrode of an electrically stimulating piece.

A further object of the present invention is to provide a low frequency assistor for electric-stimulating breast acupuncture point portions of a human body, wherein the positive electrode and the negative electrode are made as adhering pieces.

The various objects and advantages of the present invention will be more readily understood from the following detailed description when read in conjunction with the appended drawing.

### BRIEF DESCRIPTION OF THE DRAWINGS

FIG. 1 shows a partial schematic view in that at least one pair of positive electrode and negative electrode are connected serially or in parallel to a conductive film to be as an electrically stimulating piece.

FIG. 2 shows a cross sectional view of the present invention.

FIG. 3 shows a perspective view of the controller of the present invention.

FIG. 4 is a perspective view showing that the present invention is used to an inner surface of a woman brassiere.

FIG. 5 is a schematic cross sectional view of FIG. 4.

FIG. 6 shows one embodiment showing that in the present invention, the electrically stimulating piece is designed as have different positive electrode and negative electrode.

FIG. 7 shows another embodiment showing that in the present invention, the electrically stimulating piece is designed as have different positive electrode and negative electrode.

FIG. 8 is a schematic view showing that the electrically stimulating piece of the present invention is combined to an inner surface of a brassiere.

FIG. 9 is a schematic view showing the application of the present invention, wherein the electrically stimulating piece of the present invention is directly installed at an inner surface of the brassiere integrally.

FIG. 10 shows one the present invention of the present invention, wherein a controller is used to control the positive electrode and negative electrode.

FIG. 11 is a schematic view showing that a switch is installed at an outer side of a brassiere for adjusting the current of the electrically stimulating piece according to the present invention.

FIGS. 12 to 17 shows various embodiments of the present invention, where at least one pair of positive electrode and negative electrode forming an electrically stimulating piece is installed to the breast acupuncture points of human body.

FIG. 18 is a lateral schematic view includes that the conductive film of the present invention is formed as an electrically stimulating piece which is used to the cups of a woman.

FIG. 19 shows the electrically stimulating piece of the present invention forming by copper plate or conductive rubber is used to the cups of a brassiere of a woman.

FIG. 20 is a schematic view showing one embodiment of the present invention, where a wired controlled way for controlling the positive electrode and negative electrode according to the present invention.

FIG. 21 is a schematic view showing another embodiment of the present invention, where a wired controlled way for controlling the positive electrode and negative electrode according to the present invention.

FIG. 22 is a schematic view showing that the electrically stimulating piece of the present invention is designed as adhereing piece.

FIG. 23 is a schematic cross sectional view of FIG. 22.

FIG. 24 shows one application of the embodiment illustrated in FIG. 22.

### DETAILED DESCRIPTION OF THE PREFERRED EMBODIMENTS

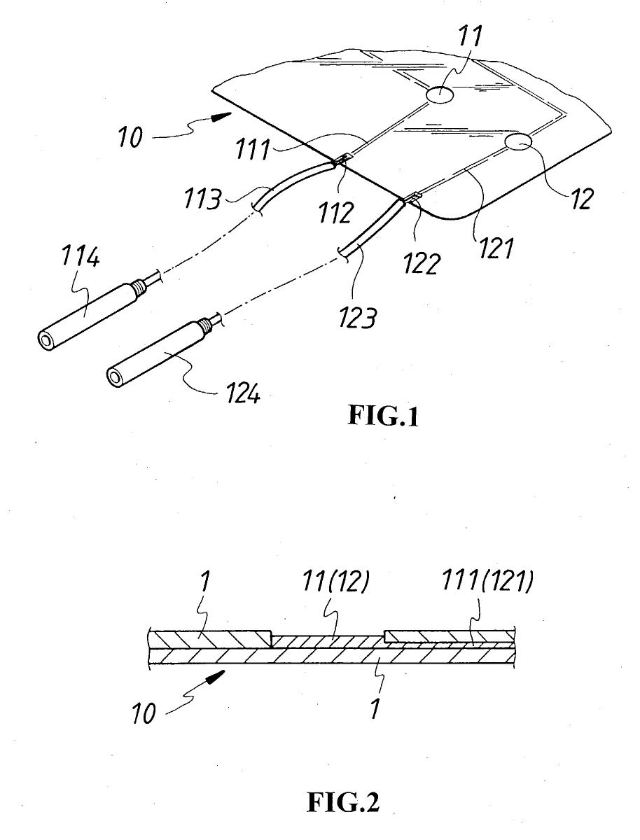

Referring to FIGS. 1 and 2, the low frequency assistor for electric-stimulating breast acupuncture point portions of a human body of the present invention is illustrated. In the present invention, at least one pair of positive electrode 11 and negative electrode 12 are connected serially or in parallel on a conductive film 1 (may be a copper plate or a conductive rubber material) so as to be formed as an electrically stimulating piece 10. Each of the positive electrode 11 and negative electrode 12 are connected to a polarity joint 112 and a polarity joint 122 through the conductive paths 111 and 121, respectively. Each of the polarity joint 112 and polarity joint 122 may be connected to the conductive pins 114 and 124 of the positive electrode 11 and the negative electrode 12, respectively. The conductive paths 111 and 121 can be installed in the thin clamping layers of the conductive film 1 so that the positive electrode 11 is insulated to the negative electrode 12.

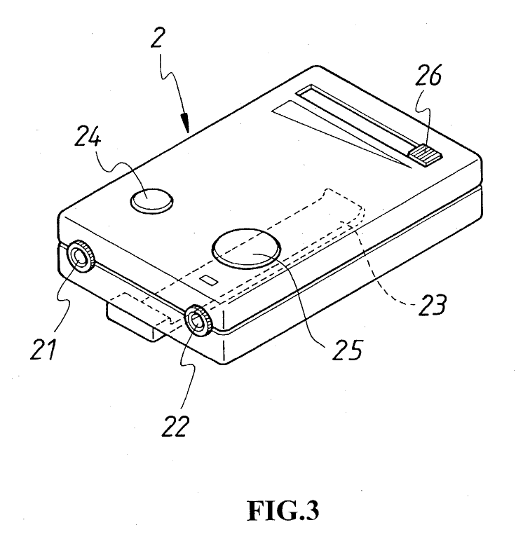

In above mentioned structure, the at least one positive electrode 11 and negative electrode 12 are connected in parallel or serially to the conductive film 1 (or copper plate or conductive rubber) to be formed as an electrically stimulating piece 10 so that the positive electrode 11 and negative electrode 12 can be applied to the breast acupuncture points. Then a wire or wireless controller 2 (see FIG. 3) is used to control the at least pair positive electrode 11 and negative electrode 12. The breast acupuncture points can be stimulated by using low frequency so that blood is circulated to present a better effect.

The controller 2 has batteries for adjusting the output current and has many selection functions. The conductive pins 21 and 22 of different electrodes are electrically connected to the conductive pins 114 and 124. The controller 2 may be installed with a clip 23 at a predetermined position as desired for being worn by users. The switches 24 and 25 serve to turn on or off the controller. The switch 26 serves to adjust the current. It may be a push-pull button or a stage-less switch.

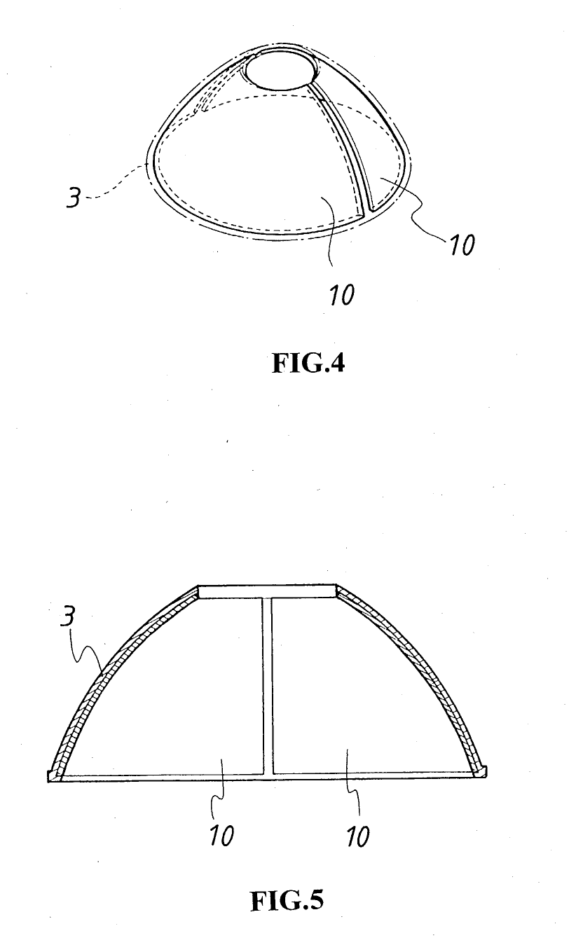

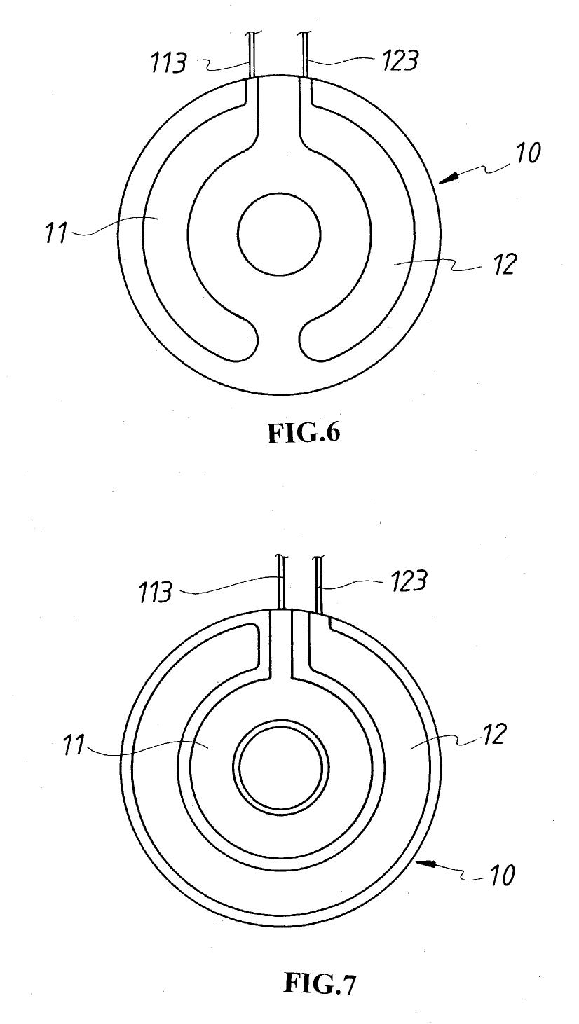

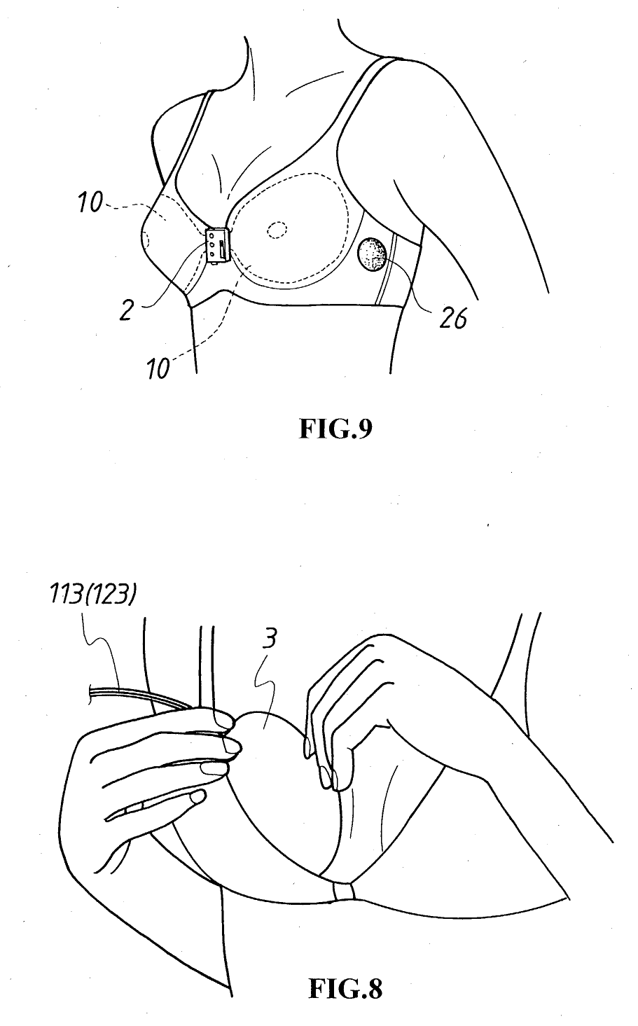

Referring to FIGS. 4 and 5, the electrically stimulating piece 10 is used in the brassiere cup 3 so that the positive electrode 11 and negative electrode 12 are positioned corresponding to the breast acupuncture points. Thereby, in conduction, the breast acupuncture points can be electrically stimulating so as to beautify the breast of the user.

In the present invention, the distribution of the positive electrode 11 and negative electrode 12 of the electrically stimulating piece 10 in the cup 3 is not confined in the present invention. Thereby, by positioning the electrically stimulating piece 10 to proper breast acupuncture points, swelling of the breast can be removed.

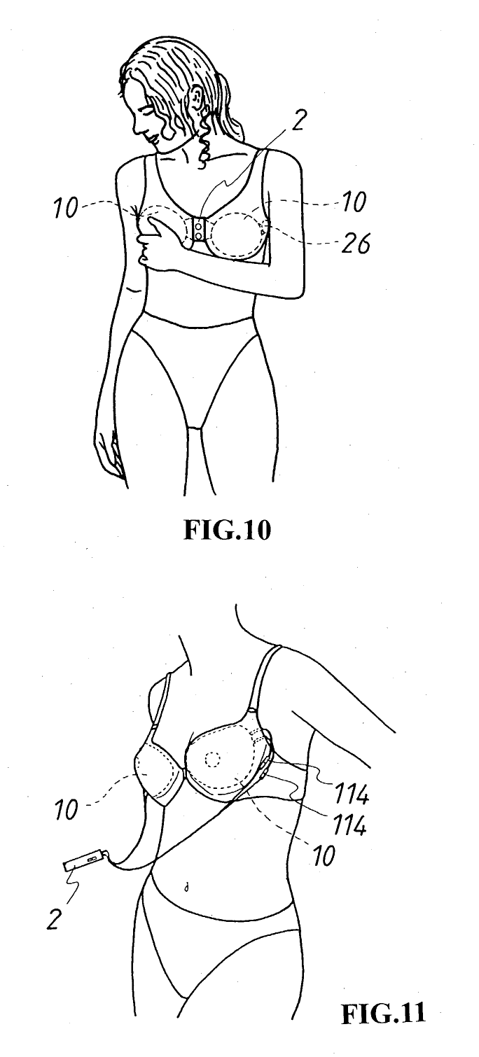

The electrically stimulating piece 10 in the cup 3 can be adhered to an inner surface of one cup 3 (referring to FIG. 9). A wired or wireless controller 2 can be used to control any set of positive electrode 11 and negative electrode 12 (referring to FIGS. 9 and 10). Moreover, the switch 26 can be installed at two sides of the brassiere for controlling the current). By low frequency assistor for electric-stimulating breast acupuncture point portions of a human body, the circulation of blood can be improved and the breast of the user can be enlarged. Moreover, the controller 2 for controlling current can be installed at an outer side of the brassiere so that it can be operated conveniently (referring to FIGS. 9 and 11).

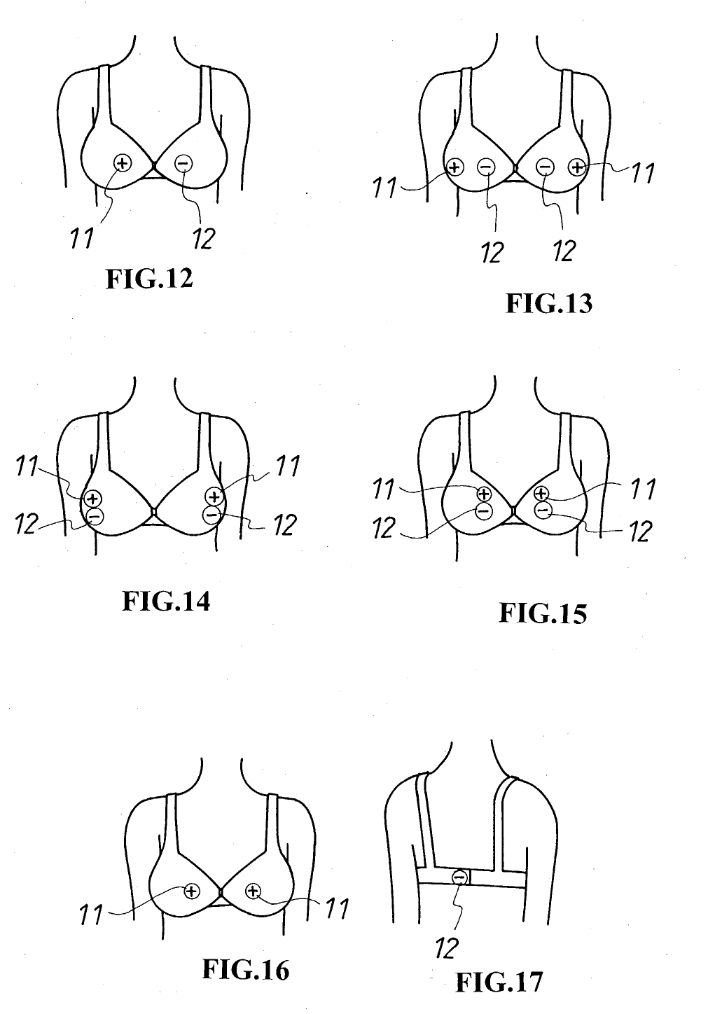

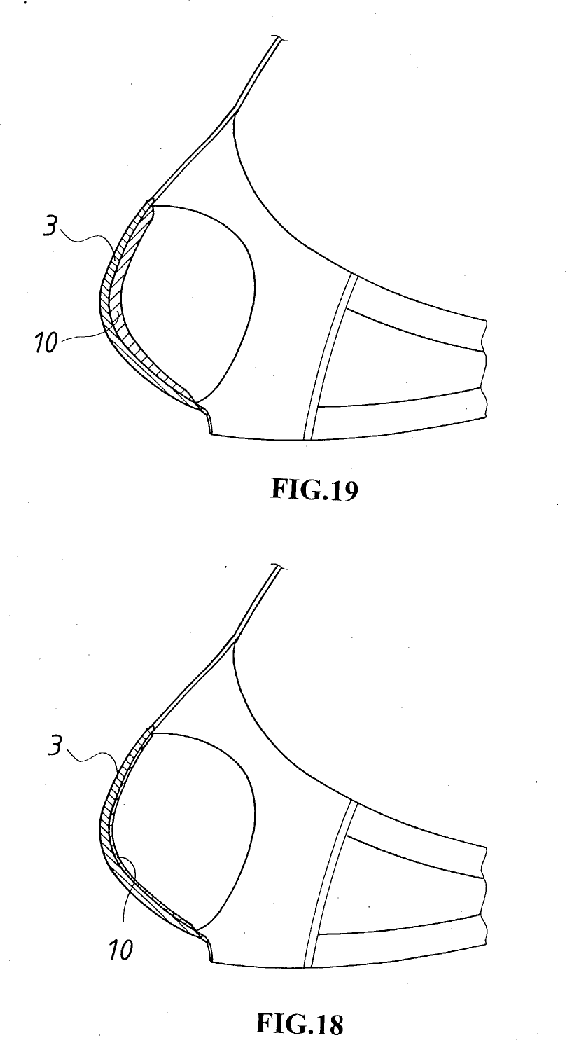

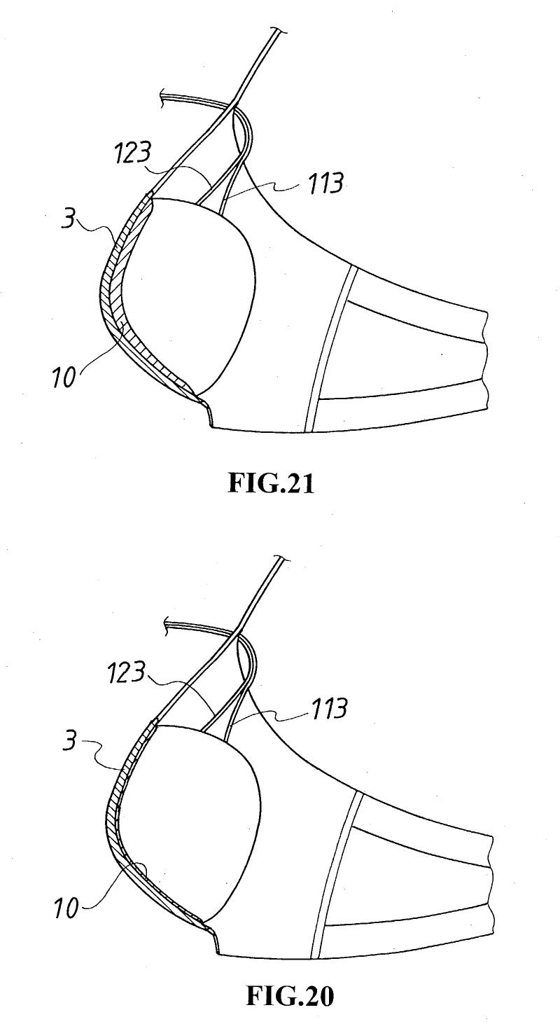

Referring to FIGS. 12 and 17, another embodiment of the present invention is illustrated. In that, at least one set of positive electrode 11 and negative electrode 12 can be distributed at the selected breast acupuncture points. The positive electrode 11 and negative electrode 12 of the electrically stimulating piece 10 in the inner surface of the cup can be controlled by wired or wireless way.

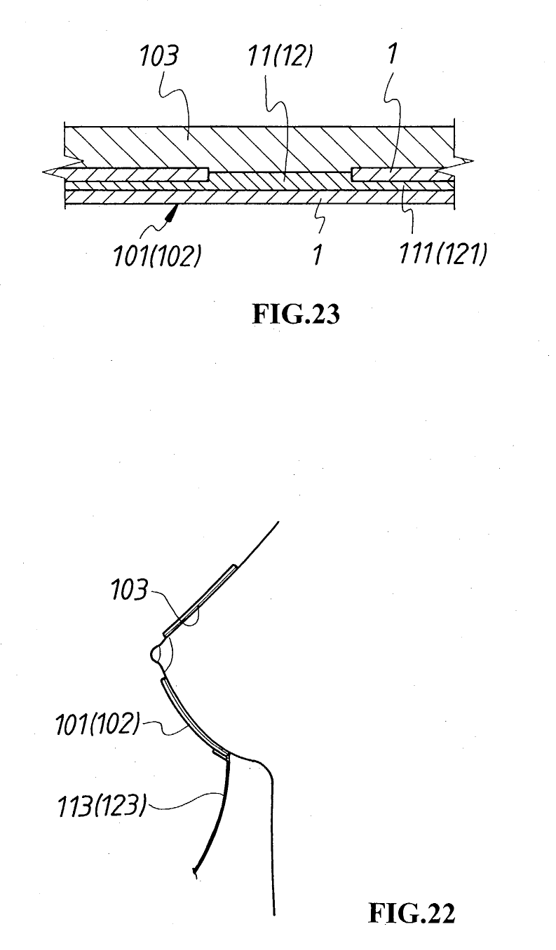

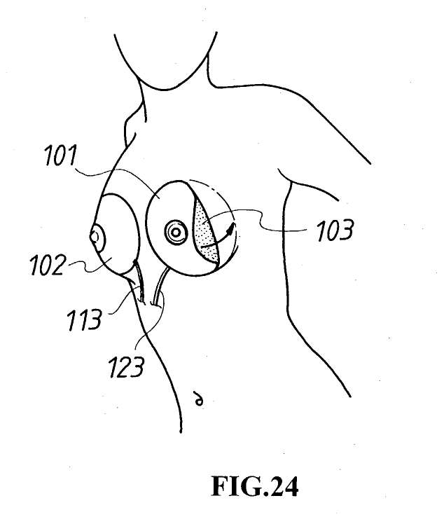

Referring to FIGS. 22 and 24, the electrically stimulating piece 10 used in the woman breast can be made as separated positive electrode piece 101 and negative electrode piece 102. Each piece is coated with a conductive silicon rubber 103 for being adhered to the breast of the woman so that low frequency massage can be performed.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A low frequency assistor for electric-stimulating breast acupuncture point portions of a human body; the device comprising at least one pair of positive electrode and negative electrode which are connected serially or in parallel on a conductive film or a copper plate or a conductive rubber material so as to be formed as an electrically stimulating piece; in application, the positive electrode and negative electrode are placed on breast acupuncture points of human body.

2. The low frequency assistor for electric-stimulating breast acupuncture point portions of a human body as claimed in claim 1, wherein each of the positive electrode and negative electrode are connected to a polarity joint and a polarity joint through different conductive paths.

3. The low frequency assistor for electric-stimulating breast acupuncture point portions of a human body as claimed in claim 2, wherein each of the positive electrode and negative electrode are connected to positive polarity joint and negarive polarity joint through the conductive paths, respectively; each of the polarity joint and polarity joint be connected to the conductive pins of the positive electrode and the negative electrode, respectively.

4. The low frequency assistor for electric-stimulating breast acupuncture point portions of a human body as claimed in claim 2, the conductive paths are installed in a thin clamping layers of the conductive film so that the positive electrode is insulated to the negative electrode.

5. The low frequency assistor for electric-stimulating breast acupuncture point portions of a human body as claimed in claim 1, wherein a wire or wireless controller is used to control the at least pair positive electrode and negative electrode of an electrically stimulating piece.

6. The low frequency assistor for electric-stimulating breast acupuncture point portions of a human body as claimed in claim 1, wherein the controller serves to control the output current and have selective functions.

7. The low frequency assistor for electric-stimulating breast acupuncture point portions of a human body as claimed in claim 1, wherein the electrically stimulating piece is installed in a cup of a brassiere and at least one pair of positive electrode and negative electrode are distributed on the breast acupuncture points of a woman.

8. The low frequency assistor for electric-stimulating breast acupuncture point portions of a human body as claimed in claim 1, wherein the electrically stimulating piece is embedded into an inner surface of a brassiere.

9. The low frequency assistor for electric-stimulating breast acupuncture point portions of a human body as claimed in claim 1, wherein the electrically stimulating piece is integral formed in an inner surface of a brassiere.

10. The low frequency assistor for electric-stimulating breast acupuncture point portions of a human body as claimed in claim 9, wherein an outer surface of the brassiere is installed with a switch for controlling current.

11. The low frequency assistor for electric-stimulating breast acupuncture point portions of a human body as claimed in claim 1, wherein the positive electrode and the negative electrode are made as adhering pieces.

12. The low frequency assistor for electric-stimulating breast acupuncture point portions of a human body as claimed in claim 11, wherein the two adhering pieces are adhered on the skin of a user and are coated with conductive silicon.
